(19) United States
(12) Patent Application Publication
(10) Pub. No.: US 2007/0140972 A1
(43) Pub. Date: Jun. 21, 2007

(54) Targeting compositions and preparation therof

(75) Inventors: Ying Zhu, Helsinki (FI); Heli Valtanen, Soderkulla (FI); Sami Kaukinen, Helsinki (FI); Oula Penate Medina, Helsinki (FI); IIkka Simpura, Helsinki (FI)

Correspondence Address: BIRCH STEWART KOLASCH & BIRCH, PO BOX 747, FALLS CHURCH, VA, 22040-0747, US

(73) Assignee: CTT CANSER TARGETING TECHNOLOGIES OY, Helsinki (FI)

(21) Appl. No.: 10/576,086

(22) PCT Filed: Oct. 15, 2004
(86) PCT No.: PCT/FI04/50150

§ 371 Date: Dec. 18, 2006

(30) Foreign Application Priority Data
Oct. 17, 2003 (FI) 20031528

(51) Int. Cl. A61K 49/10; A61K 38/12; A61K 9/127

(52) U.S. Cl. 424/9.34; 424/450; 514/9

(57) ABSTRACT

The present invention relates to targeted cancer therapy and tumour imaging, and concerns specifically new derivatives of small matrix metalloproteinase inhibitor peptides. These new derivates are the hydrophilic peptides GRENYHGCTTHWGFTLC and derivates thereof. These peptides have an increased solubility and may be used in the preparation of targeting compositions together with suitable linker molecules such as PEG. Such targeting compositions are useful in therapeutics and imaging liposome compositions for cancer treatment and diagnostics.

### FIELD OF THE INVENTION

The present invention relates to targeted cancer therapy and tumour imaging, and concerns specifically new derivatives of small matrix metalloproteinase inhibitor peptides. The peptide derivatives obtained have improved properties and may be used in the preparation of targeting compositions together with suitable linker molecules. Such targeting compositions are useful in therapeutic and imaging liposome compositions for cancer treatment and diagnostics.

### BACKGROUND OF THE INVENTION

In chemotherapy, only a fraction of the drug reaches cancer cells, whereas the rest of the drug may damage normal tissues. Adverse effects can be reduced by the administration of cancer drugs encapsulated in liposomes (Lasic et al., 1995). Improved liposome compositions have been described, so as to enhance their stability and to prolong their lifetime in the circulation (Tardi et al., 1996). Among such compositions, phospholipids conjugated to monomethoxy polyethylene glycol (PEG) have been widely used since 1984 when Sears coupled, via an amide link, carboxy PEG and purified soy phosphatidyl ethanolamine (PE) (Sears, 1984). The addition of PEG onto the liposome surface attracts a water shell surrounding the liposome. This shell prevents the adsorption of various plasma proteins (opsonins) to the liposome surface so that liposomes are not recognized and taken up by the reticulo-endothelial system. Enhanced selectivity can be obtained by attaching to the surface of the liposome specific antibodies or small peptides recognizing plasma membrane antigens of the target cell, thus augmenting the uptake of the liposome by the cell (Storm and Crommelin, 1998; Dagar et al, 2001; Peñate Medina et al., 2001).

Matrix metalloproteinases (MMPs) constitute a family of enzymes capable of degrading the basement and extracellular matrix. MMPs can be divided into subgroups, one of which constitutes the type IV collagenases or gelatinases, MMP-2 and MMP-9. Elevated or un-regulated expression of gelatinases and other MMPs can contribute to the pathogenesis of several diseases, including tumour angiogenesis and metastasis, rheumatoid arthritis, multiple sclerosis, and periodontitis. Random phage peptide libraries have been screened in order to develop a selective inhibitor against this MMP subgroup. The most active peptide derived, abbreviated CTT, was found to selectively inhibit the activities of MMP-2 and MMP-9 (Koivunen et al., 1999). Experiments in mice bearing tumour xenografts showed that CTT-displaying phages were accumulated in the tumour vasculature after their intra-venous injection into the recipient mice. Targeting of the phage to tumours was inhibited by the co-administration of the CTT peptide (Koivunen et al., 1999). As both MMP-2 (Toth et al., 1997) and MMP-9 (Brooks et al., 1996) are bound by specific cell surface receptors, these enzymes represent potential receptors for liposome targeting to invasive cells, such as tumour cells and angiogenic endothelial cells. By mixing CTT peptide with liposomes, enhanced tumour targeting and uptaking can be achieved (Peñate Medina et al., 2001).

Screening of phage display libraries allows rapid identification of peptides binding to a target. However, functional analysis of the phage sequences and their reproduction as soluble and stable peptides are often the most time-consuming parts in the screening. An intein-directed methodology can be used for synthesis and design of peptides obtained by phage display (Björklund et al., 2003). Using this technology, a library of peptide derivatives was made. A novel CTT peptide derivative (CTT2=GRENYHG-Cyclo-(CTTHWGFTLC)-NH₂) was identified. It has improved solubility in physiological solutions and is biologically active.

### SUMMARY OF THE INVENTION

We describe here various derivatives of the CTT2 peptide that can be used in cancer therapeutics and tumour imaging, and preparation thereof. CTT2 peptide and its derivatives may be covalently attached to suitable linker molecules, especially synthetic lipids. The peptide/lipid composition is purified by a specific method. The composition forms micelles in aqueous solutions and can be incorporated into liposomes. Because of the targeting properties of the peptides used, this invention creates a novel and versatile targeting tool for different types of liposomal formulations of pharmaccuticals and imaging agents. The use of the targeting tool is shown to improve the biodistribution profile and the therapeulical efficacy of the drug formulation. The peptide/lipid composition itself also has tumour imaging function in vivo. Other derivatives of the CTT2 peptide were prepared in order to improve solubility of the peptide and usefulness thereof in tumour imaging.

### BRIEF DESCRIPTION OF THE DRAWINGS

FIG. 1. Thin layer chromatography (TLC) analysis of the coupling reaction. Lane 1, CTT2 peptide control; Lane 2, DSPE-PEG-NHS control; Lane 5, the supernatant after the diethyl ether treatment; Lane 8, the pellet suspension after the diethyl ether treatment.

FIG. 2. The result of the HPLC gel filtration to separate the CTT2-PEG-DSPE compound from the CTT2 peptide. The first peak shown in the graph contains the product, CTT2-PEG-DSPE. The last peak shown in the graph contains the CTT2 peptide.

FIG. 3a. MALDI-TOF analysis of the CTT2 peptide.

FIG. 3b. MALDI-TOF analysis of the DSPE-PEG-NHS.

FIG. 3c. MALDI-TOF analysis of the CTT2-PEG-DSPE after the HPLC purification.

FIG. 4. Tumour accumulation of CTT2-coated Doxil®/Caelyx® and Doxile/Caelyx® in ovarian cancer xenograft mice over a period of 96 hours.

FIG. 5. Survival of tumour-bearing mice after the treatment with different drug/liposoine formulations.

FIG. 6. The biodistribution study of 1-125-CTT2-PEG-DSPE. The in vivo biodistribution of the ¹²⁵I-labeled micelle was assessed at two time points in NMRI/nude mice carrying human ovarian tumours on their lower back. Results are expressed as percentage of injected dose per 1 g of tissue (% ID/1 g). All values are indicated as mean±SD of 5 mice.

FIG. 7a. Molecular structure of amidated CTT2 peptide.

FIG. 7b. Molecular structure of G→K derivative of the CTT2 pcptide.

FIG. 7c. Molecular structure of G→K(DOTA) derivative of the CTT2 peptide.

FIG. 7d. Molecular structure of an indium-labeled G→K(DOTA)-CTT2 peptide.

FIG. 7e. Molecular structure of Ac-CTT2-K-NH₂ peptide.

FIG. 7f. Molecular structure of Ac-CTT2-K(DOTA)-NH₂, peptide.

FIG. 7g. Molecular structure of 6F-Trp derivative of the CTT2 peptide.

FIG. 7h. Molecular structure of 5F-Trp derivative of CTT2 peptide.

FIG. 7i. Molecular structure of 5-OH-Trp derivative of CTT2 peptide.

FIG. 8. The biodistribution study of I-125 labelled 6F-Trp CTT2 (GRENYHGCTTH[6-fluoro]WGFTLC)-peptide. The in vivo biodistribution of the ¹²⁵I-labeled peptide was assessed at two time points in NMRI/nude mice carrying human ovarian tumours on their lower back. Results are expressed as percentage of injected dose per 1 g tissue (% ID/1 g). All values are indicated as mean±SD of 5 mice.

### DETAILED DESCRIPTION OF THE INVENTION

The invention describes a hydrophilic peptide and its derivatives, which can be used in cancer therapeutics and tumour imaging, as well as a process to synthesize such peptides. In a most preferred embodiment of the invention the peptide is the cyclic CTT2 peptide having the amino acid sequence GRENYHGCTTHWGFTLC (SEQ ID NO:1), which peptide is used as an efficient targeting tool for a liposomal formulation of pharmaceuticals or imaging agents. The peptide (CTT2) is first covalently attached (coupled) to the end group of the poly(ethylene glycol) polymer chain of the PEG phospholipids, DSPE-PEG. The CTT2-PEG-DSPE suspension, which forms micelles in an aqueous solution, is then incorporated to the pre-formed liposomes that are loaded with pharmaceuticals or imaging agents. Because of the targeting properties of the CTT2 peptide and its derivatives, this invention creates a novel and versatile targeting tool for different types of liposomal formulations of pharmaceuticals and imaging agents. The use of this targeting tool is shown to improve the biodistribution profile and the therapeutical efficacy of the drug formulation. Separating the coupling and the incorporation steps makes the system versatile. The physical stress imposed on the peptide and its bond to the PEG phospholipid by conventional liposome formation procedure is avoided. The invention also describes such derivatives of the CTT2 peptide, which have improved solubility and better suitability in tumour imaging.

In principle, any peptide having suitable targeting capacity can be attached to a liposome with any composition and loaded with any substances. Consequently, the liposome can carry as a pharmaceutical a chemotherapeutic agent, e.g. doxoribicin, cisplatin or paclitaxel. The liposome can also carry an imaging agent. The peptides can be attached to suitable able nanoparticles as well.

Useful peptides having suitable targeting capacity include for instance the matrix metalloproteinase inhibitory peptides described in the international patent applications WO 99/47550 and WO 02/072618.

In specific, amidated form of the CTT2 peptide, i.e. GRENYHG-cyclo-(CTTHWGFTLC)-NH₂, and the new derivatives thereof described herein, i.e. the peptides KRENYHG-cyclo-(CTTHWGFTLC), K(DOTA)RENYHG-cyclo-(CTTHWGFTLC), K(DOTA(ln))-RENYHG-cyclo-(CTTHWGFTLC), Ac-GRENYHG-cyclo-(CTTHWGFTLC)K-NH₂, Ac-GRENYHG-cyclo-(CTTHWGFTLC)K(DOTA)-NH₂, GRENYHG-Cyclo(CTTH(d,1-6-Fluoro-W)GFTLC)-NH₂, GRENYHG-Cyclo(CTTH(d,1-5-Fluoro-W)GFTLC)-NH₂ and GRENYHG-Cyclo-(CTTH(d,l-5-OH-W)GFTLC)-NH₂ are especially suitable for the preparation of the targeting composition.

Consequently, a general object of the present invention is a targeting composition, which comprises a peptide having tumour-targeting capacity, preferably one of the above-indicated peptides, attached to a suitable lipid. The composition obtained can be used as a targeting moiety in various medical and diagnostic applications to direct a liposome to the desired target. The method of preparing such a targeting composition having tumour-targeting capacity comprises covalent attachment of a hydrophilic peptide to a synthetic derivative of polyethylene glycol.

Another object of this invention is a purification method for the targeting composition obtained by covalently attaching the cyclic GRENYHGCTTHWGFTLC peptide (CTT2 peptide) or a derivative thereof to a synthetic derivative of polyethylene glycol. In the purification method the peptide-lipid mixture obtained is incubated with an organic solvent to obtain a precipitate, the precipitate is centrifuged, washed with an organic solvent and recentrifuged to obtain a pellet, the pellet is suspended into a suitable buffer and size-exclusion chromatography is carried out to obtain pure targeting composition.

A still further object of this invention is a method for preparing a therapeutic or imaging liposome composition, comprising the steps of obtaining liposomes carrying at least one chemotherapeutic agent or imaging agent, preparing a targeting composition having tumour targeting capacity, by covalently attaching a derivative of small matrix metalloproteinase inhibitor peptide to a synthetic derivative of polyethylene glycol, and combining the liposomes and the targeting composition to form a suspension.

Still another object of the invention is a method for treating cancer in a patient, comprising the steps of obtaining liposomes carrying at least one chemotherapeutic agent, obtaining a targeting composition comprising a derivative of small matrix metalloproteinase inhibitor peptide and a synthetic derivative of polyethylene glycol, combining the liposomes and the targeting composition to form a suspension, and administering the suspension obtained to the patient.

Still another object of the invention is a diagnostic or imaging composition, comprising a targeting composition comprising a derivative of small matrix metalloproteinase inhibitor peptide and a synthetic derivative of polyethylene glycol, and liposomes carrying at least one imaging agent, or a diagnostic test kit including such a composition.

Abbreviations:

AUC Area Under Curve
CMC critical micellar concentration
CTT2 amidated cyclic GRENYHGCTTHWGFTLC peptide
DMF dimethylformamide
DOTA 1,4,7,10-tetraazacyclododecane-1,4,7,10-tetraacetic acid
Doxil®/Caelyx® commercially available doxorubicin HCl liposome injection composition by Ortho Biotech, a subsidiary of Johnson & Johnson/ Schering Plough Corporation
DSPE-PEG-NHS 1,2-Distearoyl-sn-Glycero-3-Phosphoethanolamine-n-[poly (ethylene glycol)]-N-hydroxysuccinamidyl carbonate
HPLC high-performance liquid chromatography
MMP matrix metalloproteinase
PEG poly(ethylene glycol)
RT room temperature
SL stealth liposome
TFA trifluoroacetic acid
TLC thin-layer chromatography

### EXPERIMENTAL

Peptide Coupling

In this procedure, CTT2 peptides were covalently attached to PEG phospholipids through the chemical reaction between the terminal amine of the peptide and the functional NHS (hydroxysuccinimidyl) group at the end of the poly(ethylene glycol) polymer chain of the PEG phospholipid. The reaction between the terminal amine and the active succinimidyl ester of the PEG carboxylic acid produced a stable amide linkage. Different molar ratios of the peptide and the PEG phospholipid, as well as the reaction time and temperature were tested to optimize the coupling reaction.

The pH of dimethylformamide (DMF) (BDH Laboratory Supplies) was adjusted to 8.0 by trifluoroacetic acid (TFA) (Merck). Four milligrams of synthetic amidated GRENYHG-CTTHWGFTLC peptide (CTT2) (Neosystem S.A.) and 8.6 milligrams of 1,2-distearoyl-sn-glycero-3-phosphoethanolamine-n-[poly(ethylene glycol) 3400]-N-hydroxysuccinamidyl carbonate (DSPE-PEG-NHS 3400) (Nektar Corporation) were dissolved in 1 ml DMF (pH 8.0). The mixture (molar ratio 1: 1) was incubated at +37° C. for two hours with shaking.

Purification

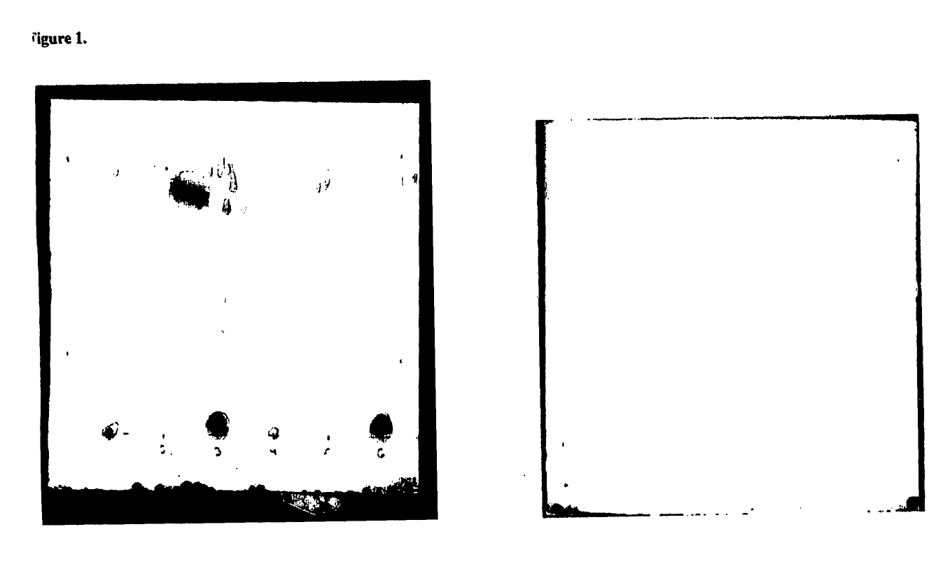

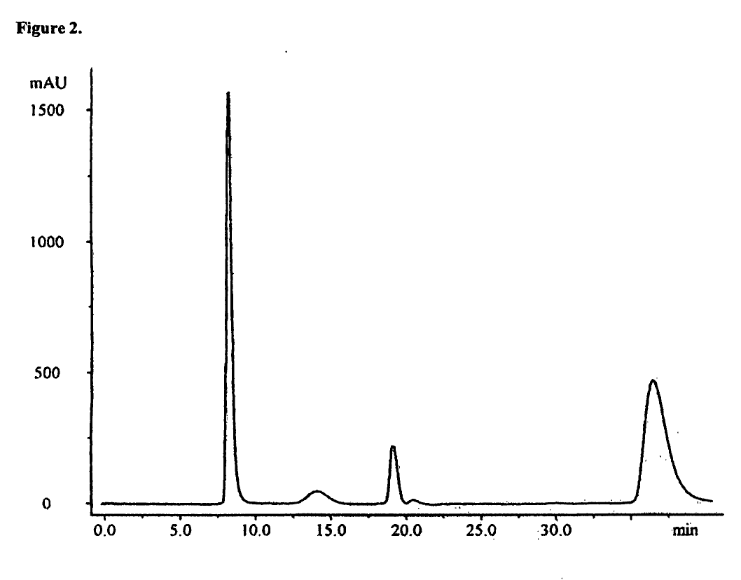

Two steps of purification were used to purify the product. First, CTT2-PEG-DSPE and CTT2 were extracted from the reaction mixture using diethyl ether (FIG. 1). Second, CTT2-PEG-DSPE was separated from CTT2 using HPLC gel filtration (FIG. 2).

The reaction mixture (1 ml) was incubated with 5 ml diethyl ether at −20° C. for 1 hour. It was then centrifuged at 13000 rpm for 10 min in a centrifuge that was pre-cooled down to +4° C. The pellet was re-suspended in 5 ml cold diethyl ether and centrifuged again. The pellet was lyophilized for 1 hour.

The pellet was dissolved in 100 μl of 50 mM ammonium acetate buffer +0.1% TFA, pH 4.5, which is the mobile phase in HPLC. Fifty microlitres of the sample were injected at a time. An isocratic run of 1 ml/min was carried out in the AKTA Purifier 10 (Amersham) with the Superdex 75 10/300 GL gel filtration column (Amersham, 1.5 ml) for 1.5 × column volume. The detection wavelength was 221 nm, with detection at wavelengths 230 and 280 nm for additional information. The fraction(s) containing the product was lyophilized, followed by the re-suspension in 400 μl of water and lyophilization again in order to remove the ammonium acetate.

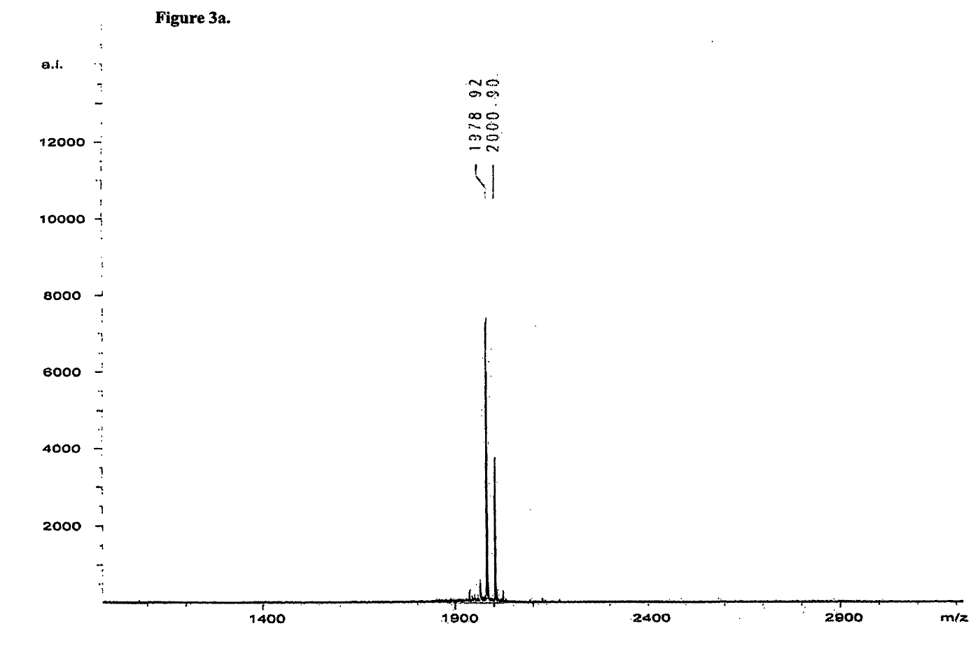

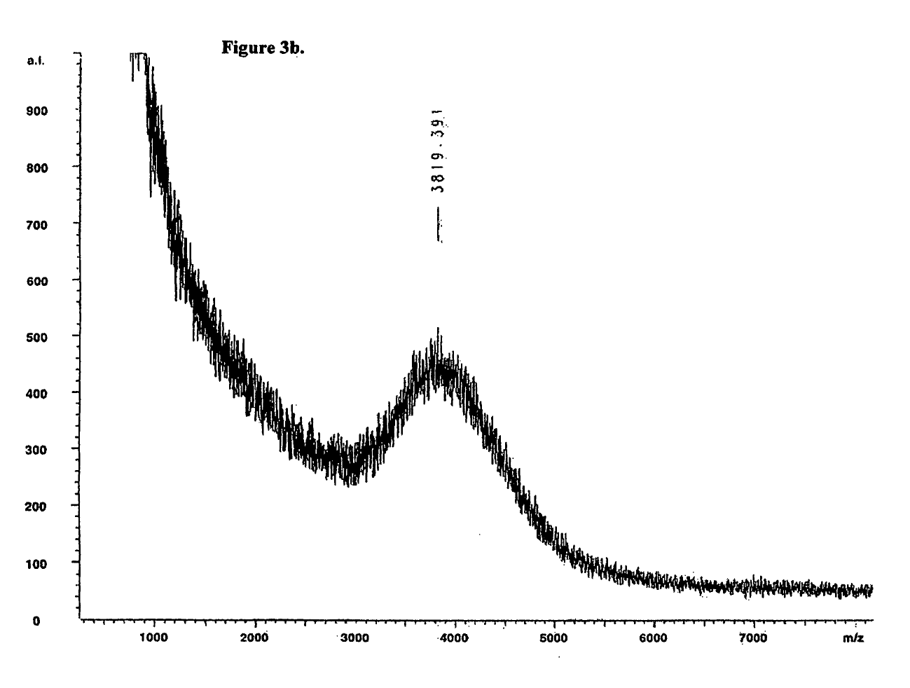

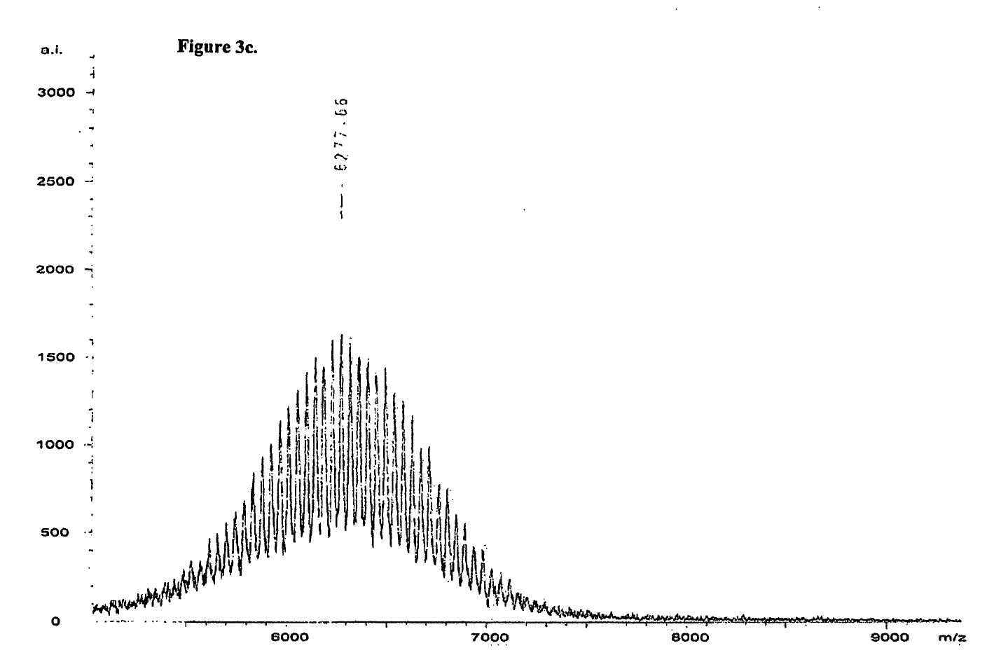

The amount of the product was measured by a modified version of the Rousell assay as described below. MALDI-TOF analysis was used to confirm the purity and the identity of the product (FIGS. 3a., 3b. and 3c.). The integrity of the cyclic structure of the CTT2 peptide was verified by the Ellman's test as described below. For long-term preservation, the lyophilized product can be preserved in dry surroundings at −20° C.

Determination of the Coupling Efficiency

Each molecule of the product CTT2-PEG-DSPE contains one molecule of phospholipid DSPE. Therefore, by measuring the concentration of the phospholipid DSPE, the concentration of the product is obtained. The phospholipid concentration was measured by a modification of the Rousell assay (Böttcher et al., 1961).

Ten microlitres of the product were added to one glass tube containing 0.2 ml of perchloric acid, and heated for 30 min at 180° C. to 190° C. To make the phosphate standard series, 0 μl, 10 μl, 25 μl, 50 μl, 75 μl, 100 μl, 150 μl, and 200 μl of 0.4 mM Na₂HPO₄ solution were added to 8 glass tubes containing 0.2 ml of perchloric acid/tube. After heating and cooling down the sample, 2 ml of molybdenate reagent (3.5 mM (NH₄)₆Mo₇O₂₄ and 1% H₂SO₄) was added to each tube containing the sample and the phosphate standard series. 0.25 ml of ascorbic acid/tube was added as well. The tubes were incubated in boiling water for 5 min and cooled down. The absorbance was measured at 812 nm. The values of the absorbance of the phosphate standard were used to make a linear regression function and the concentration of the sample was calculated using the function.

By comparing the amount of the product and the amount of the starting material, the yield of the coupling reaction can be calculated. In average, the coupling yield was around 15%. Therefore, the starting material of one milligram of CTT2 peptide and 2.05 milligrams of DSPE-PEG-NHS would produce approximately 0.5 milligrams of CTT2-PEG-DSPE.

Ellman's Test

This assay has conventionally been used for peptides (3 to 26 mer) with a single Cys residue present, but it is feasible for multiple Cys residues as well. 5,5′-dithio-bis-(2-nitro-benzoic acid) known as DNTB can be used for quantification of free sulfhydryl groups in solution. A solution of this compound produces a quantifiable yellow-coloured product when it reacts with free sulfhydryl groups to yield a mixed disulfide and 2-nitro-5-thiobenzoic acid (TNB). A sulfhydryl group can be quantified by reference to the extinction coefficients of DNTB. Sulfhydryl groups in cyclic peptides are not present, because the cysteines are linked together through S—S bonds. When a cyclic peptide is reduced, the sulfhydryl groups can be quantified with Ellman's test. This test can be used for making sure that cyclic peptide is still in active form.

The test was performed using Ellman's reagent according to the instructions of the manufacturer (Pierce). The results were measured spectrophotometrically at 412 nm. If the value was bigger than 0.020, the peptide was no longer active. Otherwise the cyclic structure of the peptide was still intact. It was shown that the coupling procedure did not disturb the cyclic structure of the CTT2-peptide. However, this test should be performed on each new batch of coupled peptide to validate the quality.

CTT2-Coated Liposomal Doxorubicin

It has been shown that the incubation of some lipids with liposomes can result in the incorporation of the lipids into the liposomes (Kanda et al., 1982). The exact mechanism is not known yet. This could happen either through the fusion of the micelle to the liposome, the micelle being formed automatically in an aqueous solution when the lipid concentration is above the critical micellar concentration (CMC), or through the exchange of phospholipids between the micelle and the liposome. As an example, we prepared the CTT2 peptide-coated liposomal doxorubicin by incorporating the CTT2-PEG-DSPE micelle with preformed liposomal doxorubicin. In the experiments we used both commercially available liposomal doxorubicin injection composition (Doxil®/Caelyx®) and liposomal doxorubicin prepared in our laboratory (data not shown). We further demonstrated the improved biodistribution profile and the therapeutic efficacy of the CTT2 peptide-coated Doxil®/Caelyx®.

CTT2-Coated Doxil®/Caelyx®

One milligram of CTT2-PEG-DSPE was suspended in 400 μl of buffer (100 mM histidine, 55 mM sucrose, pH 6.5). To 1 ml Doxil®/Caelyx® solution (Ortho Biotech), 100 μl of the CTT2-PEG-DSPE micelle suspension was added. The mixture was incubated at +60° C. for 30 min. The suspension was then ready to be injected to mice or humans. The suspension can also be preserved at +4° C. for at least 3 weeks.

The incorporation efficiency can be measured by using radioisotope-labelled peptide and gel-filtration to separate the unreacted micelle from the liposome. The incorporation efficiency is represented by the percentage of the activity in liposome fractions out of the total activity. Different incubation times and temperatures were tested, and the incubation at +60° C. for 30 min was found to be the optimal reaction conditions. The efficiency of incorporation under these conditions was close to 100%. Based on the average size and surface area of the liposomes, the amount of CTT2 peptide per liposome can be calculated. Under the reaction conditions described above, there are approximately 500 pieces of CTT2 molecules per liposome. Therefore, this amount of CTT2 peptide attached should give the liposome high enough targeting activity.

The leakage of doxorubicin from the liposomes after the incorporation experiments at different reaction times and temperatures were determined by comparing the amount of free doxorubicin before and after the experiment. The leakage was found to be minimal (the leakage before the incorporation was in average 4.5% and after the reaction in average 4.2%).

In, Vivo Studies of CTT2-Coated Doxil®/Caelyx®

In order to show the targeting capacity of the CTT2 peptide, we compared the biodistribution profiles and the therapeutic efficacies of the Doxil®/Caelyx® injection with and without the CTT2 coating. The biodistribution studies with the radioisotope-labelled CTT2 peptide were first performed on xenograft mice bearing different types of human tumours. The highest accumulation of this peptide was observed in ovarian carcinoma xenografts. Thus, the A2780 ovarian carcinoma mouse model was chosen for the subsequent biodistribution and therapy studies.

Biodistribution Studies

A2780 ovarian carcinoma cells were cultured in RPMI 1′medium (Biowhittaker) containing 10% foetal calf serum (Biowhittaker). After harvesting of the cells, 5.0×10⁶ cells were injected subcutaneously into posterior flank of 5-6-week-old NMRI nude female mice. The biodistribution study was performed when the tumour size had become about 10 mm in diameter. A2780 ovarian carcinoma-bearing mice were injected with the liposomal doxorubicin dose of 9 mg of doxorubicin/kg via a tail vein. Mice were killed 2 h, 6 h, 24 h, 48 h, 72 h and 96 h after the injection for the collection of blood, heart, liver, kidney, lung, muscle, brain, spleen and tumour samples. The blood was centrifuged at 5000 rpm for 10 min at +4° C. to obtain plasma. The tissues were frozen in liquid nitrogen and lyophilized for two days in dark. The dried tissues were weighed and extracted with acid alcohol (0.3M HCl in 50% EtOH) to obtain the final concentration of 20 mg/ml. The tissue homogenates were centrifuged at 13000 ×g for 10 min at +4° C. The cleared plasma and the cleared tissue extracts were determined for doxorubicin fluorescence using spectrofluoro-meter (Varian). Doxorubicin fluorescence was analysed by monitoring the fluorescence intensity at 590 nm using excitation wavelength of 470 nm, and comparing with standard samples containing known amounts of doxorubicin that had been processed in the same manner.

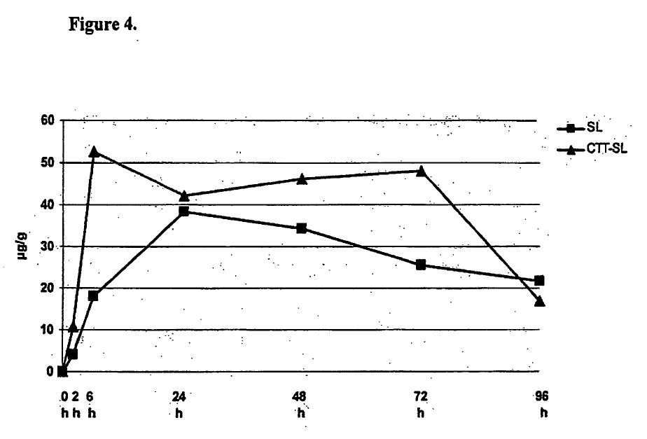

The AUC of CTT2-coated Doxil®/Caelyx (CTT-SL) accumulation in tumour was 46.2% higher than the tumour accumulation of Doxil®/Caelyx® (SL) over a period of 96 hours (FIG. 4). This shows the significant increase in the tumour targeting capacity of CTT2-coated Doxil®/Caelyx®.

Therapeutic Efficacy in Xenograft Mice

A2780 cells were injected subcutaneously into the posterior flanks of 50 NMRI nude female mice. The mice were randomly allocated into five treatment groups. To investigate the effect of different treatments on survival, the mice were treated with drugs when the tumour size had grown 5 mm in diameter (65 mm³). In this study, the mice received three drug injections of 9 mg liposomal or free doxorubicin/kg in three-day intervals. Doxorubicin concentration in CTT2-coated Doxil®/Caelyx (CTT-SL), Doxil®/Caelyx (SL) and free formulations was 2 mg/ml and thus the injection volumes varied between 120-150 l.

The mice were weighed and their tumour sizes were measured twice a week after treatment initiation. When tumour sizes exceeded 1000 mm³ the mice were sacrificed.

By five weeks after treatment initiation all mice, which were treated with buffer, with CTT2-micelle or with free doxorubicin had been sacrificed and only 33% of Doxil®/Caelyx-treated mice were alive. However, at the same time 75% of CTT2-coated Doxil®/Caelyx-treated mice were still alive (FIG. 5). Mean survival time for CTT2-coated Doxil®/Caelyx group was 38.6 days and for Doxil®/Caelyx 27.9 days.

Biodistribution of CTT2-PEG-DSPE

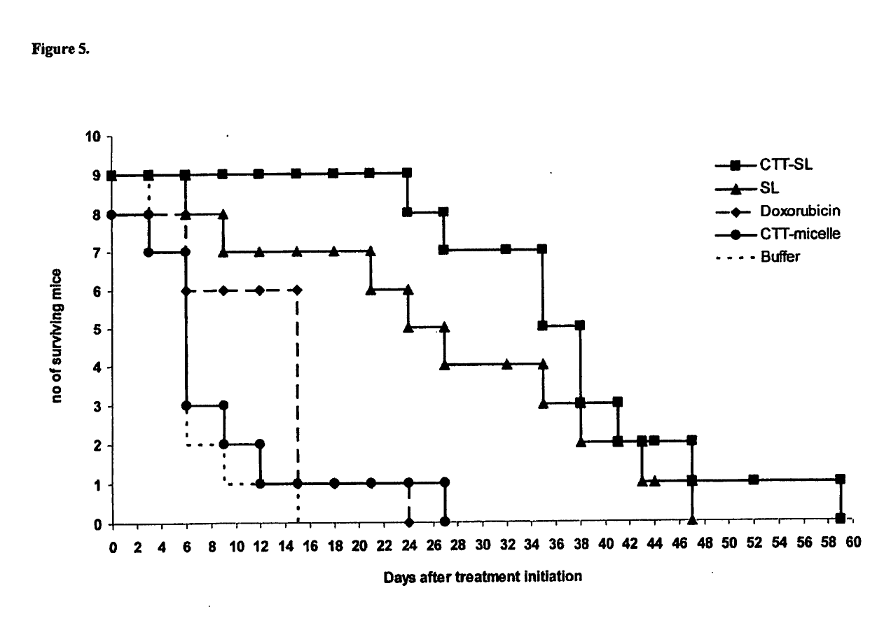

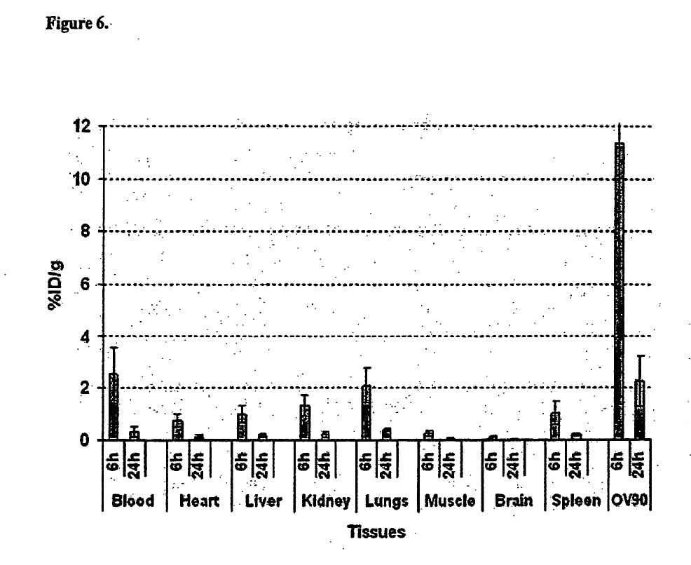

CTT2-PEG-DSPE was produced as described above. To study the tissue distribution of the CTT2-Peg3400-DSPE molecule in cancer xenograft model, ten immunodeficient mice were inoculated with human ovarian carcinoma cells (OV-90). When the tumour xenograft grafts were fully established (about three weeks after implantation), the biodistribution study was performed by injecting iodine-labelled CTT2-PEG-DSPE (200 μg; ˜1 MBq) in 200 μl PBS into the tail vein of mice. At 6 h and 24 h post injection, the mice were sacrificed and their blood and tissues were dissected for gamma counting. Highest accumulation of radioactivity was observed in tumour xenografts at both time points studied (tumour/muscle ratio 43) (FIG. 6.).

Derivatives of the CTT2 Peptide

CTT2 can be viewed as having two structurally distinct parts. Cyclic (-CTTHWGFTLC) part of the peptide is more hydrophobic compared to the linear GRENYHG- part of the peptide. The attachment point (N-terminus vs. C-terminus) of CTT2 peptide to any molecular moiety might have effect on conjugate solubility and bioactivity. Two different peptide derivatives (peptides 1 and 4 in Table 1) were synthesized in order to improve the solubility and bioactivity of conjugates.

The peptides can be used as probes for in vivo imaging of physiological states and processes. CTT2 peptide can be directly labelled with radioactive iodine. More sophisticated radioactive imaging agents, e.g. ¹¹¹ In and ^{99m}Tc require a chelator moiety conjugated to original peptide. DOTA derivatives of CTT2 peptide (peptides 2, 3 and 5 in Table 1) were synthesized, and one of them (peptide 3 in Table 1) was labelled with cold indium. These peptide-DOTA conjugates (peptides 2 and 5 in Table 1) can be labelled with radioactive isotopes to be used either in diagnostic (¹¹¹In ) or therapeutic purposes (¹⁷⁷Lu, ⁹⁰Y).

By synthetic incorporation of an unnatural fluorotryptophan amino acid, we obtained two CTT2-peptide derivatives, 6F-Trp CTT2 and 5F-Trp CTT2 (peptides 6 and 7 in Table 1). The 6F-Trp CTT2 showed enhancement in serum stability and improved ability to inhibit tumour cell migration in comparison to the wild type peptide (see Biodistribution of the 6F-Trp CTT2 peptide). Also a 5-OH-Trp derivative was prepared (peptide 8 in Table 1).

The peptides were synthesized with an Applied Biosystems model 433A (Foster City, Calif.) using Fmoc-chemistry as reported previously (Koivunen et al., 1999), except that the disulfide bond formation was conducted using hydrogen peroxide.

Briefly, the peptide was dissolved in 50 mM ammonium acetate (pH 7.5) at a 1 mg/ml concentration and 0.5 ml of 3% hydrogen peroxide per 100 mg peptide was added. After 30 min incubation, pH was adjusted to 3.0 and the cyclized peptide was purified by reverse-phase HPLC using a linear acetonitrile gradient (0%-70% during 30 min) in 0.1% trifluoroacetic acid.

Indium labelling of DOTA derived peptide: 1.2 mg of K(DOTA)RENYHG-cyclo-(CTTHWGFTLC) was dissolved in 100 μl of ammonium acetate buffer (pH 6.5). InCl₃ was dissolved in ammonium acetate buffer (pH 6.5). Two molar equivalents of InCl₃ solution were added to the peptide solution. Reaction mixture was left standing overnight at RT. Indium-labelled peptide was purified by reverse phase C-18 cartridges using ammonium acetate buffer (pH 6.5) and acetonitrile solution (50%/50%). Indium-labelled peptides were obtained as white solid after lyophilization of freezed eluates. Indium-labelled peptides were identified by MALDI-TOF MS.

TABLE 1

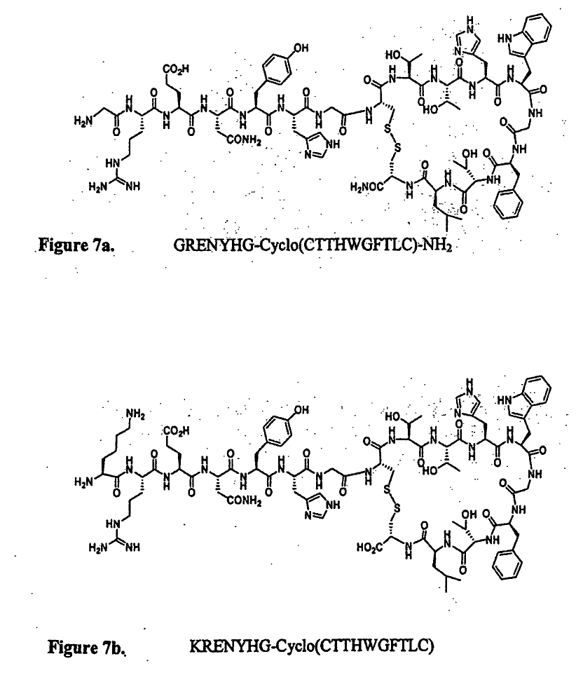

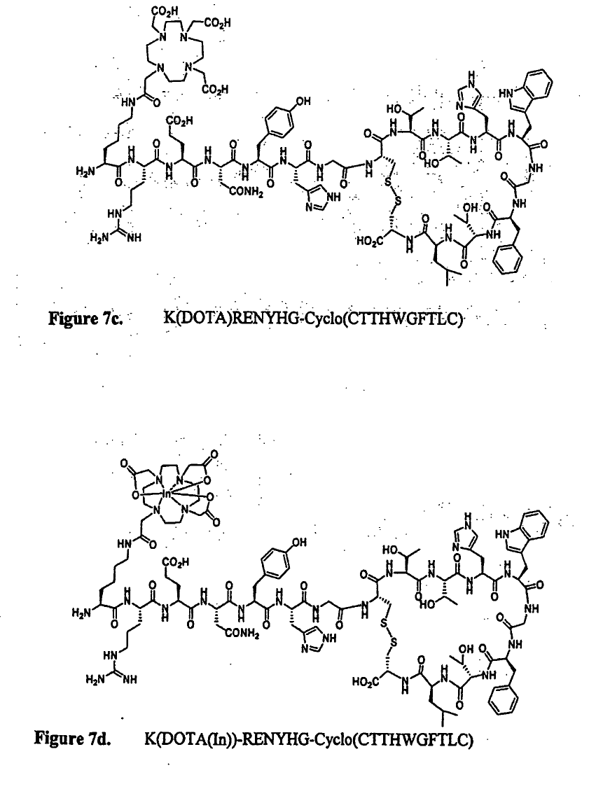

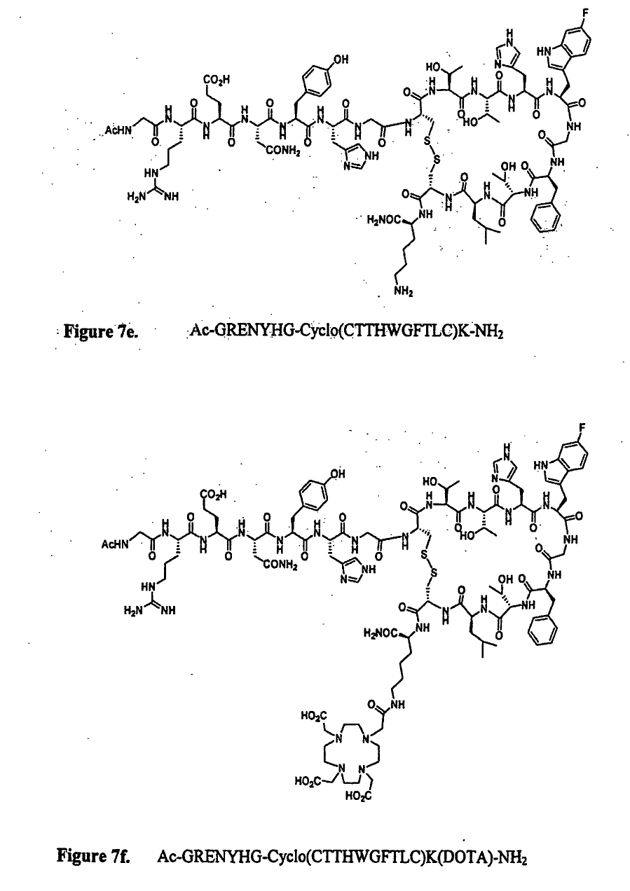

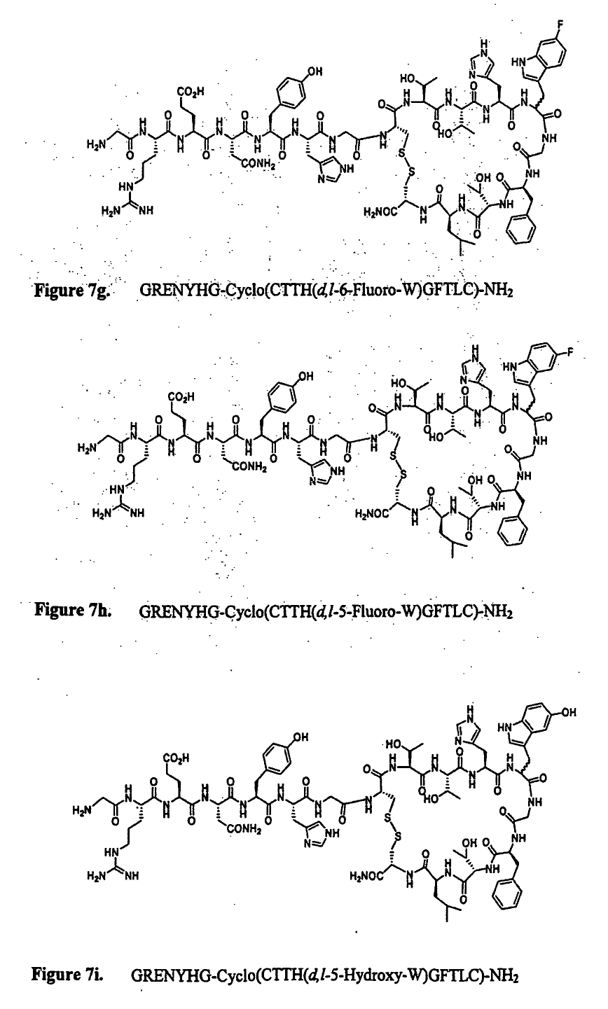

Derivatives of CTT2 peptide (see FIGS. 7b to 7i
for the molecular structures)

| | Exact | Observed |
|---|---|---|
| | mass | mass |
| Peptide sequence | (M)/g/mol | (M + H⁺)/g/mol |
| (1) KRENYHG-cyclo-(CTTHWGFTLC) | 2049, 89 | 2050, 91 |
| (2) K(DOTA)RENYHG-cyclo-(CTTHWGFTLC) | 2436, 07 | 2436, 99 |
| (3) K(DOTA(ln))RENYHG-cyclo-(CTTHWGFTLC) | 2547, 95 | 2548, 69 |
| (4) Ac-GRENYHG-cyclo-(CTTHWGFTLC)K-NH₂ | | |
| (5) Ac-GRENYHG-cyclo-(CTTHWGFTLC)K(DOTA)-NH₂ | | |
| (6) GRENYHG-Cyclo(CTTH(d,l-6-Fluoro-W)GFTLC)-NH₂ | 1995, 83 | 1996, 77 |
| (7) GRENYHG-Cyclo(CTTH(d,l-5-Fluoro-W)GFTLC)-NH₂ | 1995, 83 | |
| (8) GRENYHG-Cyclo(CTTH(d,l5-OH-W)GFTLC)-NH₂ | 1995, 83 | |

Biodistribution of the 6F-Trp CTT2 peptide

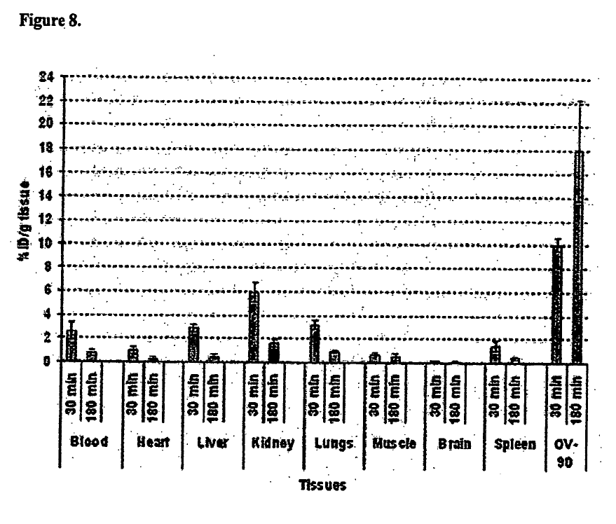

The 6F-Trp CTT2 peptide was used in biodistribution study to evaluate its kinetic and tumour targeting properties. The study was performed in mice with established human ovarian carcinoma tumours (OV-90). The 6F-Trp CTT2 peptide was labelled with iodine-125. 40 μg of purified and labelled peptide (˜1 MBq) was injected into the tail vein of mice. 30 min and 180 min after peptide injection mice were sacrificed and blood and tissue samples were collected. The accumulated radioactivity was determined with gamma counter. The results showed a remarkable accumulation of radioactivity in tumour tissue with tumour/muscle ratios 14.9 and 23.3 at 30 min and 180 min, respectively. Instead, in all other organs the accumulation of radioactivity was negligible and the clearance was comparable to blood (FIG. 8). The possibility of using unnatural amino acids in peptide synthesis may provide more active and stable peptides for tumour targeting.

### REFERENCES

Björklund, M., Valtanen, H., Savilahti, H., and Koivunen, E. Use of intein-directed peptide biosynthesis to improve serum stability and bioactivity of a gelatinase inhibitory peptide. Comb Chem High Throughput Screen 6:29-35, 2003.

Brooks, P., Stromblad, S., Sanders, L., von Schalscha, T., Aimes, R., Stetler-Stevenson, W., Quigley, J., and Cheresh, D. Localization of matrix metalloproteinase MMP-2 to the surface of invasive cells by interaction with integrin αᵥβ₃- Cell 85: 683 -693, 1996.

Böttcher, C. J. F.,Van Gent, C. M., Pries, C. Anal. Chim. Acta 24: 203-204, 1961.

Dagar, S., Sekosan, M., Lee, B., Rubinstein, I., and Önyüiksel, H. VIP receptors as molecular lar targets of breast cancer: implications for targeted imaging and drug delivery. Journal of Controlled Release 74: 129-134, 2001.

Kanda, S., Inoue, K., Nojima, S., Utsumi, H., and Wiegandt, H. Incorporation of ganglioside and spin-labelled ganglioside analogue into cell and liposome membranes. J. Biochem. 91: 2095-2098, 1982.

Koivunen, E., Arap, W., Valtanen, H., Raininsalo, A., Peñ ate Medina, O., Heikkikä, P., Kantor, C., Gahmberg, C., Salo, T., Konttinen, Y., Sorsa, T., Ruoslahti, E., and Pasqualini, R. Cancer therapy with a novel tumour-targeting gelatinase inhibitor selected by phage peptide display. Nature Biotechnol. 17: 768-774, 1999.

Lasic, D., Ceh, B., Stuart, M., Guo, L., Frederik, P., and Barenholz, Y. Transmembrane gradient driven phase transitions within vesicles: lessons for drug delivery. Biochim. Biophys. Acta 1239: 145-156, 1995

Peñate Medina, O., Soderlund, T., Laakkonen, L., Tuominen, E., Koivunen, E., and Kinnunen, P. Binding of novel peptide inhibitors of type IV collagenases to phospholipid membranes and use in liposome targeting to tumour cells in vitro. Cancer Res. 61: 2978-2985, 2001.

Sears, B. D. (1984). Synthetic Phospholipid Compounds. U.S. Pat. No. 4,426,330.

Storm, G., and Crommelin, D. Liposomes: quo vadis? Pharm. Sci. & Tech. Today 1: 19-31, 1998.

Tardi, P., Boman, N., and Cullis, P. Liposoinal doxorubicin. J. Drug Target. 4: 129-140, 1996.

Toth, M., Gervasi, D., and Fridman, R. Phorbol ester-induced cell surface association of matrix metalloproteinasc-9 in human MCFIOA breast epithelial cells. Cancer Res. 57: 3159-3167,1997.

1. A method of preparing a targeting composition having tumour-targeting capacity, comprising covalently attaching the cyclic GRENYHGCTTHWGFTLC peptide (CTT2 peptide) (SEQ ID NO: 1) or a derivative thereof to a synthetic derivative of polyethylene glycol.

2. The method according to claim 1, wherein the synthetic derivative of polyethylene glycol is DSPE-PEG.

3. The method according to claim 2, wherein the DSPE-PEG is DSPE-PEG-NHS.

4. The method according to claim 1, wherein the derivative of the CTT2 peptide is a peptide selected from the group consisting of KRENYHG-cyclo-(CTTHWGFTLC) (SEQ ID NO: 2), K(DOTA)RENYHG-cyclo-(CTTHWGFTLC) (SEQ ID NO: 2), K(DOTA(In))RENYHG-cyclo-(CTTHWGFTLC) (SEQ ID NO: 2), Ac-GRENYHG-cyclo-(CTTHWGFTLC)K-NH₂ (SEQ ID NO: 3), Ac-GRENYHG-cyclo-(CTTHWGFTLC)K(DOTA)-NH₂ (SEQ ID NO: 3), GRENYHG-Cyclo(CTTH(d,l-6-Fluoro-W)GFTLC)-NH₂(SEQ ID NO: 4), GRENYHG-Cyclo(CTTH(d,l-5-Fluoro-W)GFTLC)-NH₂ (SEQ ID NO: 4) and GRENYHG-Cyclo-(CTTH(d,l-5-OH-W)GFTLC)-NH₂ (SEQ ID NO: 4).

5. The method according to claim 4, wherein the synthetic derivative of polyethylene glycol is DSPE-PEG-NHS.

6. A method for preparing a therapeutic or imaging liposome composition, comprising the steps of
(a) obtaining liposomes carrying at least one chemotherapeutic agent or an imaging agent,
(b) preparing a targeting composition having tumour-targeting capacity, by covalently attaching the cyclic GRENYHGCTTHWGFTLC peptide (CTT2 peptide) (SEQ ID NO: 1) or a derivative thereof to a synthetic derivative of polyethylene glycol, and
(c) combining the liposomes and the targeting composition to form a suspension.

7. The method according to claim 6, wherein the derivative of the CTT2 peptide is a peptide selected from the group consisting of KRENYHG-cyclo-(CTTHWGFTLC) (SEQ ID NO: 2), K(DOTA)RENYHG-cyclo-(CTTHWGFTLC) (SEQ ID NO: 2), K(DOTA(In))RENYHG-cyclo-(CTTHWGFTLC) (SEQ ID NO: 2), Ac-GRENYHG-cyclo-(CTTHWGFTLC)K-NH₂ (SEQ ID NO: 3), Ac-GRENYHG-cyclo-(CTTHWGFTLC)K(DOTA)-NH2 (SEQ ID NO: 3), GRENYHG-Cyclo(CTTH(d,l-6-Fluoro-W)GFTLC)-NH₂ (SEQ ID NO: 4), GRENYHG-Cyclo(CTTH(d,l-5-Fluoro-W)GFTLC)-NH₂ (SEQ ID NO: 4) and GRENYHG-Cyclo-(CTTH(d,l-5-OH-W)GFTLC)-NH₂ (SEQ ID NO: 4).

8. A method for treating cancer in a patient, comprising the steps of
(a) obtaining liposomes carrying at least one chemotherapeutic agent,
(b) obtaining a targeting composition comprising
(1) the cyclic GRENYHGCTTHWGFTLC peptide (CTT2 peptide) (SEQ ID NO: 1) or a derivative thereof and
(2) a synthetic derivative of polyethylene glycol,
(c) combining the liposomes and the targeting composition to form a suspension, and
(d) administering the suspension obtained to the patient.

9. The method according to claim 8, wherein the derivative of CTT2 peptide is a peptide selected from the group consisting of KRENYHG-cyclo-(CTTHWGFTLC) (SEQ ID NO: 2), K(DOTA)RENYHG-cyclo-(CTTHWGFTLC) (SEQ ID NO: 2), K(DOTA(In))RENYHG-cyclo-(CTTHWGFTLC) (SEQ ID NO: 2), Ac-GRENYHG-cyclo-(CTTHWGFTLC)K-NH₂ (SEQ ID NO: 3), Ac-GRENYHG-cyclo-(CTTHWGFTLC)K(DOTA)-NH₂ (SEQ ID NO: 3), GRENYHG-Cyclo(CTTH(d,l-6-Fluoro-W)GFTLC)-NH₂ (SEQ ID NO: 4), GRENYHG-Cyclo(CTTH(d,l-5-Fluoro-W)GFTLC)-NH₂ (SEQ ID NO: 4) and GRENYHG-Cyclo-(CTTH(d,l-5-OH-W)GFTLC)-NH₂ (SEQ ID NO: 4).

10. The method according to any one of claims 6 to 9, wherein the chemotherapeutic agent is doxorubicin.

11. A diagnostic or imaging test kit for carrying out a diagnostic method for detecting a suspected tumour in a patient, wherein the test kit comprises
a targeting composition comprising the cyclic GRENYHGCTTHWGFTLC peptide (CTT2 peptide) (SEQ ID NO: 1) or a derivative thereof and a synthetic derivative of polyethylene glycol, and
liposomes carrying at least one imaging agent.

12. The test kit according to claim 11, wherein the derivative of CTT2 peptide is a peptide selected from the group consisting of KRENYHG-cyclo-(CTTHWGFTLC) (SEQ ID NO: 2), K(DOTA)RENYHG-cyclo-(CTTHWGFTLC) (SEQ ID NO: 2), K(DOTA(In))RENYHG-cyclo-(CTTHWGFTLC) (SEQ ID NO: 2), Ac-GRENYHG-cyclo-(CTTHWGFTLC)K-NH₂ (SEQ ID NO: 3), Ac-GRENYHG-cyclo-(CTTHWGFTLC)K(DOTA)-NH₂ (SEQ ID NO: 3), GRENYHG-Cyclo(CTTH(d,l-6-Fluoro-W)GFTLC)-NH₂ (SEQ ID NO: 4), GRENYHG-Cyclo(CTTH(d,l-5-Fluoro-W)GFTLC)-NH₂ (SEQ ID NO: 4) and GRENYHG-Cyclo-(CTTH(d,l-5-OH-W)GFTLC)-NH₂ (SEQ ID NO: 4).

13. A diagnostic or imaging composition, comprising
a targeting composition comprising the cyclic GRENYHGCTTHWGFTLC peptide (CTT2 peptide) (SEQ ID NO: 1) or a derivative thereof and a synthetic derivative of polyethylene glycol, and
liposomes carrying at least one imaging agent.

14. The composition according to claim 13, wherein the derivative of CTT2 peptide is a peptide selected from the group consisting of KRENYHG-cyclo-(CTTHWGFTLC) (SEQ ID NO: 2), K(DOTA)RENYHG-cyclo-(CTTHWGFTLC) (SEQ ID NO: 2), K(DOTA(In))RENYHG-cyclo-(CTTHWGFTLC) (SEQ ID NO: 2), Ac-GRENYHG-cyclo-(CTTHWGFTLC)K-NH₂ (SEQ ID NO: 3), Ac-GRENYHG-cyclo-(CTTHWGFTLC)K(DOTA)-NH₂ (SEQ ID NO: 3), GRENYHG-Cyclo(CTTH(d,l-6-Fluoro-W)GFTLC)-NH₂ (SEQ ID NO: 4), GRENYHG-Cyclo(CTTH(d, 1-5-Fluoro-W)GFTLC)-NH₂ (SEQ ID NO: 4) and GRENYHG-Cyclo-(CTTH(d,l-5-OH-W)GFTLC)-NH₂ (SEQ ID NO: 4).

15. Use of a preparation comprising as a suspension
(1) a targeting composition, which comprises
(a) the cyclic GRENYHGCTTHWGFTLC peptide (CTT2 peptide) (SEQ ID NO: 1) or a derivative thereof and covalently attached thereto
(b) a synthetic derivative of polyethylene glycol, and
(2) liposomes carrying at least one chemotherapeutic agent, for the manufacture of a pharmaceutical composition useful for the treatment of cancer.

16. Use according to claim 15, wherein the derivative of CTT2 peptide is a peptide selected from the group consisting of KRENYHG-cyclo-(CTTHWGFTLC) (SEQ ID NO: 2), K(DOTA)RENYHG-cyclo-(CTTHWGFTLC) (SEQ ID NO: 2), K(DOTA(In))RENYHG-cyclo-(CTTHWGFTLC) (SEQ ID NO: 2), Ac-GRENYHG-cyclo-(CTTHWGFTLC)K-NH₂ (SEQ lD NO: 3), Ac-GRENYHG-cyclo-(CTTHWGFTLC)K(DOTA)-NH₂ (SEQ ID NO: 3), GRENYHG-Cyclo(CTTH(d,1-6-Fluoro-W)GFTLC)-NH2 (SEQ ID NO: 4), GRENYHG-Cyclo(CTTH(d,l-5-Fluoro-W)GFTLC)-NH₂ (SEQ ID NO: 4) and GRENYHG-Cyclo-(CTTH(d,l-5-OH-W)GFTLC)-NH₂ (SEQ ID NO: 4).

17. A peptide selected from the group consisting of KRENYHG-cyclo-CTTHWGFTLC) (SEQ ID NO: 2), K(DOTA)RENYHG-cyclo-(CTTHWGFTLC) (SEQ ID NO: 2), K(DOTA(In))RENYHG-cyclo-(CTTHWGFTLC) (SEQ ID NO: 2), Ac-GRENYHG-cyclo-(CTTHWGFTLC)K-NH₂ (SEQ ID NO: 3), Ac-GRENYHG-cyclo-(CTTHWGFTLC)K(DOTA)-NH₂ (SEQ ID NO: 3), GRENYHG-Cyclo(CTTH(d,l-6-Fluoro-W)GFTLC)-NH₂ (SEQ ID NO: 4), GRENYHG-Cyclo(CTTH(d,l-5-Fluoro-W)GFTLC)-NH₂ (SEQ ID NO: 4) and GRENYHG-Cyclo-(CTTH(d,l-5-OH-W)GFTLC)-NH₂ (SEQ ID NO: 4).

18. A process for purifying the targeting composition obtainable by covalently attaching the cyclic GRENYHGCTTHWGFTLC peptide (CTT2 peptide) (SEQ ID NO: 1) or a derivative thereof to a synthetic derivative of polyethylene glycol, the process comprising the steps of
(a) treating the reaction mixture with an organic solvent to obtain a precipitate,
(b) centrifuging, washing with an organic solvent and recentrifuging the precipitate to obtain a pellet,
(c) suspending the pellet in a buffer and
(d) carrying out size-exclusion chromatography to obtain pure targeting composition.

19. The process according to claim 18, wherein the organic solvent in steps (a) and (b) is diethyl ether and the buffer in step (c) is ammonium acetate—TFA buffer, pH 4.5.


SEQUENCE LISTING (4 sequences)

SEQ ID NO: 1 - protein, 17 aa, Artificial Sequence
  CTT2-peptide
GRENYHGCTTHWGFTLC

SEQ ID NO: 2 - protein, 17 aa, Artificial Sequence
  Lys-substitution of the CTT2-peptide
KRENYHGCTTHWGFTLC

SEQ ID NO: 3 - protein, 18 aa, Artificial Sequence
  CTT2-peptide with additional Lys
GRENYHGCTTHWGFTLCK

SEQ ID NO: 4 - protein, 17 aa, Artificial Sequence
  CTT2-peptide with a tryptophan analogue at position 12
  misc_feature (12)..(12): Xaa at position 12 is 5-OH-Trp, 5-F-Trp or 6-F- Trp
GRENYHGCTTHXGFTLC